(19) United States
(12) Patent Application Publication
(10) Pub. No.: US 2008/0248468 A1
(43) Pub. Date: Oct. 9, 2008

(54) Method for Improved Selection of Rnai Transfectants

(76) Inventors: Manfred Kubbies, Penzberg (DE); Robert Macek, Benediktbeuern (DE); Carola Ries, Penzberg (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/792,902

(22) PCT Filed: Dec. 13, 2005
(86) PCT No.: PCT/EP2005/013343

§ 371 Date: Jun. 11, 2007

(30) Foreign Application Priority Data
Dec. 14, 2004 (EP) 04029505.7

(51) Int. Cl. C12Q 1/68; C12N 15/87; C12N 15/63; C12N 5/06; C07K 16/00

(52) U.S. Cl. 435/6; 435/440; 435/320.1; 435/325; 530/388.2

(57) ABSTRACT

The present invention is directed to a method for inactivation of expression of a gene in a eucaryotic cell comprising (i) transfection of a eucaryotic cell with DNA comprising an expression cassette for expression of a cell surface protein and an expression cassette for expression of a RNAi compound, said compound being capable of inactivating expression of said gene, wherein said expression cassette for expression of a cell surface protein and said expression cassette for expression of a RNAi compound are located on the same vector DNA, and (ii) enrichment and/or selection of cells which express said cell surface protein.

The present invention relates to the field of gene inactivation by means of RNAi. More precisely, the present invention relates to the field of selection principles for enrichment and isolation of cell populations with a respective RNAi mediated gene silencing effect. In particular, the present invention is applicable for functional gene analysis using RNAi for transient and long-term silencing of gene expression in the field of oncology and apoptosis.

### BACKGROUND PRIOR ART

The phenomenon of RNAi mediated gene silencing has been described first in the Caenorhabditis elegans system, in which microinjection of long double stranded RNA molecules was reported to result in an inactivation of the respective gene (U.S. Pat. No. 6,506,559). Later on, RNAi mediated gene silencing has been disclosed in vertebrates (EP 1 114 784), mammals and in particular human cells (EP 1 144 623). In these systems, gene inactivation is achieved successfully, if short, double stranded RNA molecules of 19-29 bp were transfected in order to transiently knock down a specific gene of interest.

The mechanism of RNA mediated gene inactivation seems to be slightly different in the various organisms that have been investigated so far. In all systems, however, RNA mediated gene silencing is based on a post-transcriptional degradation of the target mRNA induced by the endonuclease Argonaute2 which is part of the so called RISC complex (WO 03/93430). Sequence specificity of degradation is determined by the nucleotide sequence of the specific antisense RNA strand loaded into the RISC complex.

Appropriate possibilities of introduction include transfecting the double stranded RNA molecule itself or in vivo expression of DNA vector constructs which directly result in a short double stranded RNA compound having a sequence that is identical to a part of the target RNA molecule. In many cases, so called shRNA constructs have been used successfully for gene silencing. These constructs encode a stem-loop RNA, characterized in that after introduction into cells, it is processed into a double stranded RNA compound, the sequence of which corresponding to the stem of the original RNA molecule.

Identification of stably transduced or transfected cells comprising an shRNA construct has already been performed by means of coexpressing a cell surface marker such as CD-4 (Barton, G. M., and Medzhitov, R., Proc. Natl. Acad. Science USA 99 (2002) 14943-14945). However, prior to the present invention, coexpression of a cell surface marker has not been used for enrichment or selection of cells expressing an artificially introduced shRNA.

Selection of transfected cells including cells transfected with RNAi expression constructs can be obtained with different methods. Most commonly, the vectors contain eucaryotic antibiotic resistance genes such as the hygromycin resistance gene or the neomycin resistance gene. Another method for detection, enrichment and at least partial selection of transfected cells is the usage of expression cassettes encoding a surface antigen which is either not or to a significant lower extent expressed in the host cells.

Subsequently, enrichment of cells may be obtained by flow cytometric methods. One prominent example is the employment of a form of the low-affinity nerve growth factor receptor 1-NGFR as surface marker to identify transfected cells (Machl, A. W., et al., Cytometry 29 (1997) 371-374).

Besides antibiotic selection methods which usually require several weeks, living cells transfected with silencing vectors containing a shRNA expression construct so far have been enriched and selected using an additional expression construct for expressing Enhanced Green Fluorescent Protein (EGFP) as an in vivo active fluorescent marker protein. To allow analysis of transiently transfected/transduced cells, vectors co-expressing shRNAs and enhanced green fluorescent protein (EGFP) have been explored (Kojima, S., et al., Biotechniques 36 (2004) 74-79). Cells expressing EGFP may also be enriched by flow cytometric methods.

However, usage of EGFP as a selection marker has several draw backs: First, EGFP overexpression may exert cytotoxic effects in transfected cells, with varying degree depending on the cell type used. Second, since the fluorescence signal of EGFP largely depends on the protein's conformation, which is perturbed by various fixation techniques, this strategy is restricted to applications alleviating fixation of cells. Third, in cases where silencing of a target gene induces cell death, EGFP is not a useful marker, as cells with damaged membranes fail to retain the soluble EGFP (Chalfie, M., and Kain, S., (eds), in Green Fluorescent Protein: properties, applications and protocols, Wiley-Liss, New York, 1998, and Harvey, K. J., et al., Cytometry43 (2001) 273-278).

Therefore, it was an object of the present invention to provide an improved method for the selection of cells transfected/transduced with RNAi constructs. In one aspect, it was an object of the present invention to provide an improved RNAi method for rapid enrichment and selection of cells transfected with RNAi constructs. In particular, it was an object of the present invention to provide an improved method for RNAi mediated gene silencing which allows the analysis of apoptotic processes in a stage of transient expression.

### BRIEF DESCRIPTION OF THE INVENTION

Thus, the present invention provides methods, compositions and kits for an improved and accelerated enrichment and selection of cells transfected with RNAi expression constructs.

In a first aspect, the present invention is directed to a method for inactivation of expression of a gene in a eucaryotic cell comprising
a) transforming a eucaryotic cell with DNA comprising an expression cassette for expression of a cell surface protein and an expression cassette for expression of a RNAi compound, said compound being capable of inactivating expression of said gene, wherein said expression cassette for expression of a cell surface protein and said expression cassette for expression of a RNAi compound are located on the same vector DNA,
b) enrichment and/or selection of cells which express said cell surface protein.

Preferably, said RNAi compound is a RNA with a hairpin conformation.

Also preferably, said enrichment and selection of cells which express said cell surface protein is performed by means of cell sorting.

In a second aspect, the present invention is directed to a composition comprising
a) a transfection reagent,
b) DNA comprising an expression cassette for expression of a cell surface protein and an expression cassette for expression of a RNAi compound, said compound being capable of inactivating expression of a gene, wherein said expression cassette for expression of a cell surface protein and said expression cassette for expression of a RNAi compound are located on the same vector DNA.

Preferably, said composition carries only one type of vector DNA. Thus, said expression cassette for expression of a cell surface protein and said expression cassette for expression of a RNAi compound are located on the same vector DNA.

In a third aspect, the present invention is directed to a vector comprising an expression cassette for expression of 1-NGFR and an expression cassette for expression of a RNAi compound, said compound being capable of inactivating expression of a gene in a eucaryotic cell.

In a fourth aspect, the present invention is directed to a kit comprising
a) a vector comprising an expression cassette for expression of a cell surface protein and an expression cassette for expression of a RNAi compound, said compound being capable of inactivating expression of a gene,
b) a monoclonal antibody or a polyclonal antiserum directed against said cell surface protein.

In a specific embodiment, said antibody is labeled with a detectable entity or covalently bound to a solid support.

In another specific embodiment, not mutually exclusive with the one described above, the kit according to the present invention further comprises a transfection reagent.

### DETAILED DESCRIPTION OF INVENTION

As disclosed above, the present invention is directed to a method for inactivation of expression of a gene in a eucaryotic cell comprising
a) transfection of a eucaryotic cell with DNA comprising an expression cassette for expression of a cell surface protein and an expression cassette for expression of a RNAi compound, said compound being capable of inactivating expression of said gene, wherein said expression cassette for expression of a cell surface protein and said expression cassette for expression of a RNAi compound are located on the same vector DNA, and
b) enrichment and/or selection of cells which express said cell surface protein.

In the context of the present invention, the term “inactivation of expression of a gene” shall be defined as degradation of a specific target RNA which is mediated by a RNAi compound. The RNAi compound itself is synthesized after transfection with an expression cassette for expression of a RNAi compound or a precursor of a RNAi compound which is subsequently processed into a RNAi compound by endogenous cellular nucleases.

In a preferred embodiment, said inactivation of gene expression is a transient inactivation. In the context of the present invention, transient inactivation is being defined as inactivation of expression of a certain gene within a population of cells without applying selective pressure. In this context, enrichment of cells coexpressing the introduced cell surface marker offers the advantage that respective analytical assays can be performed within a few days after the transfection itself. Moreover, transient transfection assays avoid problems of generating stable transformants associated with effects due to growth disadvantages that result from integration events into the host genome.

In another embodiment, in order to achieve a permanent inactivation, enrichment with a recombinantly expressed cell surface marker facilitates and in many cases accelerates isolation of stable transfectants.

Within a very short period of time RNAi has proven to be a highly specific and powerful tool to reveal gene function, and is therefore extensively used for target validation in academia and the pharmaceutical industry. So far, two major gene silencing strategies have emerged for in vitro studies: small interfering RNAs (siRNAs) and small hairpin RNAs (shRNAs) (Tuschl, T., Nat. Biotechnol. 20 (2002) 446-448). The use of chemically synthesized siRNAs is hampered by a strong dependency on rather high transfection efficiencies, which limits its applicability to well transfectable cell lines. Nevertheless, results still can vary between experiments due to different transfection efficiencies, and are only transient in nature. In contrast, vector-derived shRNAs outperform siRNAs in the duration of the achieved gene silencing effect. Moreover, they provide all advantages of a vector-based expression system such as combination with reporter genes or selection markers, and delivery via viral vectors (Brummelkamp, T. R., and Bernards, R., Nat. Rev. Cancer 3 (2003) 781-789). Thus, shRNA expression vectors overcome restrictions based on low transfection efficiency. Most of the work published on these vectors is based on cell lines, in which expression of a target gene is silenced due to stable expression of shRNAs directed against this gene (Caplen, N. J., Gene Ther. 11 (2004) 1241-1248). However, generation of stable cell clones resembles a tedious and lengthy process.

The RNAi compound according to the present invention may be a shRNA which is expressed by an appropriate expression cassette and comprises a stem of preferably 19 to 29 nucleotides in length, whose sequence is identical/complementary to the target mRNA that has to be inactivated. Alternatively, the sense and antisense strand of the RNAi compound may be transcribed from one DNA fragment with opposite promoters separately. Still alternatively, the sense and antisense strand of the RNAi compound may become transcribed from two independent DNAs encoding RNAs of appropriate lengths and sequences.

As disclosed above, the present invention combines the concept of gene silencing by means of a RNAi compound with the concept of using a cell surface protein as a selection marker. A reporter gene expressed on the cell surface can overcome the restrictions associated with the use of EGFP such as toxicity and lack of utility for analysis of apoptosis. 1-NGFR, a truncated form of the low-affinity nerve growth factor receptor, and thus inactive for signal transduction, is expressed on the cell surface and has proven to be a highly useful marker for cell biological analysis (Phillips, K., et al., Nat. Med. 2 (1996) 1154-1156 and Machl, A. W., et al., Cytometry 29 (1997) 371-374). Combining this valuable reporter, l-NGFR, with a shRNA vector (termed pHC_vector), successfully allows transient and high-content multiparametric analysis of silenced cells independent of transfection efficiency.

It is within the scope of the present invention to use any kind of gene whose expression product is located on the cell surface as a marker for enrichment and selection of transfectants expressing a high level of RNAi compound, provided that they fulfill the following three requirements:
a) The respective host cell should not express a corresponding endogenous protein at a high level.
b) Expression of the surface marker should not result in an alteration of the normal signal transduction pathways of the host cell.
c) In addition, these cell surface proteins are truncated for their intracellular signaling domains. Examples for such truncated cell surface proteins are 1-NGFR, H-2K and CD4 (Milteny: Biotec: MACSelect Transfected Cell Selection User Manual).

The present invention is applicable in general in all living cells expressing the so-called double-strand RNA nuclease Dicer and the RISC complex or, in other words in all cells where RNA mediated gene silencing can be observed. Thus, the present invention can be applied for all types of eucaryotic cells from plants, fungi, invertebrate cells, and vertebrate cells. Preferably, the present invention is applicable for mammalian cells and in particular human cells or cell lines.

Within the scope of the present invention, cell transformants may be obtained with substantially any kind of transfection method known in the art. For example, the vector DNA may be introduced into the cells by means of electroporation or microinjection. Alternatively, lipofection reagents such as FuGENE 6 (Roche Diagnostics), X-tremeGENE (Roche Diagnostics), and Lipofectamine™ (Invitrogen) may be used. Still alternatively, the vector DNA comprising expression cassettes for a cell surface protein and a RNAi compound may be introduced into the cell by appropriate viral vector systems based on retroviruses, lentiviruses, adenoviruses or adeno-associated viruses (Singer, O., Proc. Natl. Acad. Sci. USA 101 (2004) 5313-5314).

The DNA comprising the expression cassettes according to the invention may be either a circular plasmid vector or a linear DNA fragment such as a restriction fragment or, preferably a PCR product.

The DNA to be transfected may comprise either one or two independent vector DNAs. In one embodiment, the expression cassettes for expression of a cell surface protein and expression of a RNA compound are located on different DNA vectors. In a preferred embodiment, the expression cassette for expression of a cell surface protein and the expression cassette for expression of a RNA compound are located on the same vector. In this case, it needs to be avoided that the two transcripts do interfere with each other. Yet, having both expression cassettes located on the same vector DNA is highly advantageous, because it facilitates the subsequent selection of transformants expressing the RNAi compound by means of cell sorting on the basis of the expressed cell surface marker.

In the context of the present invention, the term “expression cassette” is defined as a genetic construct comprising a promoter sequence, a DNA sequence encoding the RNA that is desired to become expressed within the host cell, and, facultatively, a terminator sequence useful for terminating transcription.

The expression cassette for the expression of a cell surface protein comprises DNA encoding the cell surface protein itself, and a polymerase II (Pol II) promoter which provides for steady/constitutive expression of the cell surface protein. Examples for suitable strong promoters are the CMV promoter, the SV40 promoter and the PGK promoter. Preferably, the expression cassette also comprises a SV40 terminator element which provides for accurate termination of the Pol II transcript.

The transcript or transcripts which are constituting the RNAi compound can be either transcribed from Pol II promoters such as the CMV promoter or from a Pol III promoter like the Hi, U6 or 7SI promoters. In case of a Pol III mediated transcription, it is essential to have a Pol III terminator sequence of TTTT at the 3′ end of the transcribed RNA for appropriate 3′ processing of the precursor RNA product (Dykxhoorn, D., et al., Nat. Rev. Mol. Cell Biol. 4 (2003) 457-467).

In one preferred embodiment, the RNAi compound is a RNA with a hairpin conformation, i.e. a shRNA. As an active RNAi compound, such a molecule may start with a G nucleotide at its 5′end, due to the fact that transcription from the HI and U6 promoter usually starts with a G. The stem of the molecule is usually 19 to 29 base pairs and preferably 19 to 23 base pairs in length. The internal loop of the molecule is a single stranded chain of 4-40 and preferably 4-9 nucleotides. At the 3′end, there may be an overhang. In case of usage of a Pol III promoter, the overhang may be UUUU due to the terminator signal of Pol III promoters. When expressed within a cell, these hairpin constructs are rapidly processed into active double stranded molecules capable of mediating RNA gene silencing (Dylxhoorn, D., et al., Nat. Rev. Mol. Cell Biol. 4 (2003) 457-467).

In another preferred embodiment, the promoter for transcribing a RNAi compound may be an inducible promoter, characterized in that the promoter activity can be switched on by an external signal stimulation. Examples for such promoter systems which in principle may be combined with any kind of promoter are the tet-repressor system, hormone-inducible promoter systems (Harvey, D. M., and Caskey, C. T., Curr. Opin. Chem. Biol. 2 (1998) 512-518), tissue specific promoter or the CRE system (Ventura, A., et al., Proc. Natl. Acad. Sci. USA 101 (2004) 10380-10385). Mostly preferred is the combination of a Pol III promoter with an appropriate tet-repressor system (Invitrogen: BLOCK-iT Inducible H1 RNAi Entry Vector User Manual).

In addition, it is possible to provide a third expression cassette with confers an antibiotic resistance to the transformed cells such as for example, hygromycin or neomycin resistance.

In the context of the present invention the term “enrichment” shall comprise any method of separating a population of cells which have undergone a transfection step into two subpopulations, wherein the first subpopulation has a higher percentage of transfected cells than the second subpopulation.

In the context of the present invention the term “selection of cells” shall mean any method, wherein a population of cells that have undergone a transfection procedure is subsequently enriched for cells which actually have been transfected.

A person skilled in the art will understand that the above definitions are overlapping to a certain extent. Thus, enrichment and selection of cells in many cases may be achieved by the same methods disclosed herein.

Within the scope of the invention, transfected cells may be identified microscopically with a labeled monoclonal antibody or polyclonal antiserum which is directed against an extracellular epitope of the expressed cell surface protein marker.

Within the scope of the present invention, enrichment and selection of transfected cells may be achieved by flow cytometric tools. After labeling of the cells with a fluorescently labeled monoclonal antibody or polyclonal antiserum directed against the cell surface marker, labeled cells might either be counted or enriched by a fluorescence activated cell sorter.

In an other embodiment of the present invention, enrichment or selection of transfected cells is based on a principle, wherein a surface coated with an antibody directed against the expressed cell surface protein is used in order to bind only those cells that have been transfected successfully. Preferably, appropriate beads comprising a covalently bound monoclonal antibody or polyclonal antiserum are used as a solid support. In a very effective embodiment, transfected cells are enriched and selected by a technique called “magnetic activated cell sorting” (MACS), a readily applicable and commercially available method without the need for laborious expensive and time consuming cell sorting methods and instruments.

A detailed description of the technique is given in the MACSelect transfected cell selection user manual (Milteny: Biotec: MACSelect Transfected Cell Selection User Manual). Briefly, a cell population that has undergone a transfection procedure is incubated with magnetic beads that are coated with an antibody that binds to a surface marker which is expressed in transfected cells due to an introduced expression cassette. Thus, exclusively transfected cells which express the surface marker are capable of binding to the microbeads. Subsequently, the sample comprises a mixture of transfected cells labeled with microbeads as well as non-transfected cells, which is loaded onto a magnetic separator or respective separation column. Cells labeled with these paramagnetic beads in contrast to non-transfected cells selectively bind to the magnetic column material. By removing the magnetic field labeled cells are released from the column.

Therefore in a major aspect, the present invention is directed to an improved method of enriching and/or selecting RNAi transformants by means of using an appropriate cell surface marker. Usage of a cell surface marker allows for the immediate enrichment, and selection of transfected cells within a cell population, which express an RNAi compound in order to silence a specific gene of interest.

Usually, selection by means of an appropriate cell surface protein can be performed already after 16-48 hours, as soon as expression of the cell surface marker occurs. As a consequence, biochemical and phenotypical analysis of cells silenced for target gene expression can be studied rapidly after transfection, which excludes any potential long term artifacts when the cells are grown over multiple generations in tissue culture. Furthermore, selection by means of using an appropriate cell surface protein marker can be repeated several times.

In another aspect the present invention is directed to vectors comprising an expression cassette for expression of a 1-NGFR and an expression cassette for expression of a RNAi compound, said compound being capable of inactivating expression of a gene. Preferably, said expression cassette for expression of a RNAi compound provides a stem-loop hairpin RNA, of which the stem may act as a gene silencing mediating compound. Vectors encoding the 1-NGFR reporter in conjunction with a shRNA expression cassette enable for the first time quantitative multiparametric analysis of cells silenced for individual target gene expression. By employing 1-NGFR based shRNA pHC_vectors, various cell lines and primary cells can be analyzed for cell cycle distribution, induction of apoptosis, cellular architecture as well as for mRNA and protein expression independent of transfection efficiency.

In still another aspect, the present invention is directed to a composition comprising
a) a transfection reagent,
b) DNA comprising an expression cassette for expression of a cell surface protein, which is preferably 1-NGFR, and an expression cassette for expression of a RNAi compound, said compound being capable of inactivating expression of a gene, wherein said expression cassette for expression of a cell surface protein and said expression cassette for expression of a RNAi compound are located on the same vector DNA.

Preferably, said transfection reagent is a lipofection reagent such as FuGENE 6, X-tremeGENE (both Roche Diagnostics) or Lipofectamine™ (Invitrogen).

Also preferably, said composition carries only one type of vector DNA.

In a further aspect, the present invention is directed to a kit comprising
a) a vector comprising an expression cassette for expression of a cell surface protein, which is preferably 1-NGFR, and an expression cassette for expression of a RNAi compound, said compound being capable of inactivating expression of a gene,
b) a monoclonal antibody or a polyclonal antiserum directed against said cell surface protein.

The expression cassette for expression of a RNAi compound comprises a promotor which may be a Pol II promoter or a Pol III promoter, a cloning site to insert a specific sequence which finally will act as gene silencing mediating RNAi compound and, preferably, a respective terminator sequence.

In a specific embodiment, said antibody is labeled with a detectable entity such as a fluorescent entity or covalently bound to a solid support. For example, said solid support may be a bead, preferably a magnetic bead.

In another specific embodiment, not mutually exclusive with the one described above, the kit according to the present invention comprises several different vectors with different expression cassettes encoding different cell surface proteins. This will allow the customer to select the appropriate enrichment and selection system for the specific cell line that shall be transfected.

It is also within the scope of the present invention, if the kit comprises a transfection reagent, preferably a lipofection reagent, and one or several vector DNAs comprising an expression cassette for expression of a cell surface protein and an expression cassette for expression of a RNAi compound, said compound being capable of inactivating expression of said gene.

In addition, such a kit may also comprise a monoclonal antibody or a polyclonal antiserum directed against said cell surface protein. Facultatively, said antibody is labeled with a detectable entity such as a fluorescent entity or covalently bound to a solid support. For example, said solid support may be a bead, preferably a magnetic bead.

The following examples, references, sequence listing and figures are provided to aid the understanding of the present invention, the true scope of which is set forth in the appended claims.

### DESCRIPTION OF THE FIGURES

FIG. 1 Cloning strategy for the generation of a vector encoding both 1-NGFR and U6 promoter driven shRNA expression cassette.

FIG. 2 Knock-down efficiency of HER2 protein that is expressed on the cell surface.

FIG. 3 Enrichment of 1-NGFR-positive cells by magnetic cell separation (MACS) in transient transfection experiments.

FIG. 4 Knock-down efficiency in magnetic sorted compared to non-sorted cells and phenotypic alterations induced by silencing of cytoplasmic protein eg5.

FIG. 5 Phenotypic alterations induced by silencing of cytoplasmic protein eg5 in transient transfection assays.

FIG. 6 Multiparametric analysis of phenotypic alterations induced by silencing of eg5 in transient transfection assays.

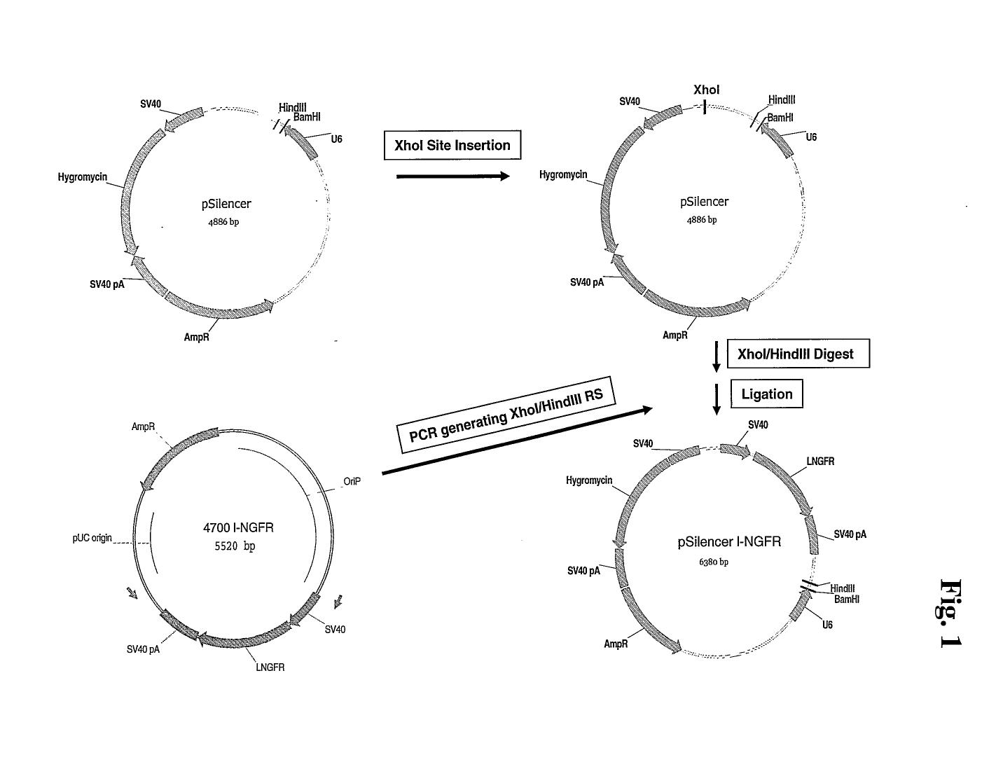

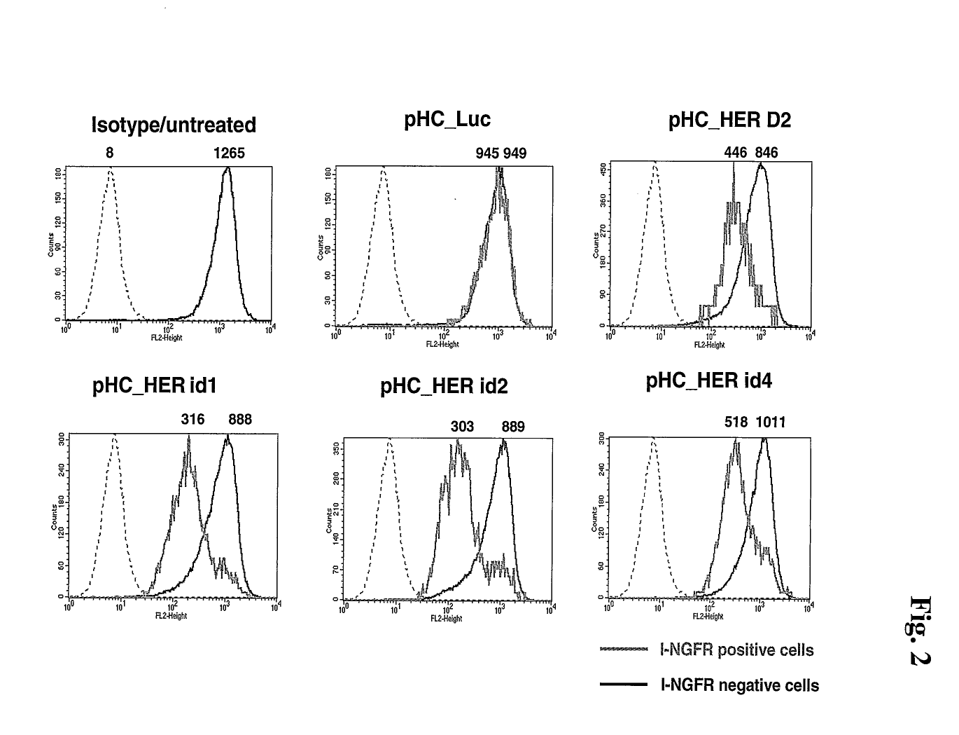

### EXAMPLE 1

Cloning Strategy for the Generation of a Vector Encoding Both 1-NGFR and a U6 Promoter Driven shRNA Expression Cassette

To generate a 1-NGFR vector coexpressing the U6 promoter driven shRNA cassette termed pHC for high-content, the 1-NGFR ORF in context with a SV40 early promoter and polyadenylation sequence was amplified from a 1-NGFR expression vector by PCR using the primers

(SEQ ID NO: 1)

pHC_FW 5′-CGCCTCGAGTCCCTGTGGAATGTGTGTCAGTTAG-3′
and

(SEQ ID NO: 2)

pHC_RV 5′-CGCAAGCTTGCTGGCCTTTTGCTCACATGTTC-3′

The 1-NGFR PCR-fragment was subcloned into pSilencer U6 2.1 Hygro (Ambion) using the HindIII and a unique restriction site for XhoI generated by site-directed mutagenesis (Stratagene). Subsequently, a variety of DNA oligonucleotides (chemically synthesized by Metabion (Germany)) encoding shRNA sequences were annealed and ligated into pHC to generate pHC_luc, pHC_eg5 and pHC_Her2:

luc:

(SEQ ID NO: 3)

5′-GATCCGCTTACGCTGACTTCGATTCAAGAGATCGAAGTACTCAGCGT
AAGTTTTTTGGAA-3′

(SEQ ID NO: 4)

5′-AGCTTTTCCAAAAAACTTACGCTGAGTACTTCGATCTCTTGAATCGA
AGTACTCAGCGTAAGCG-3′
eg5:

(SEQ ID NO: 5)

5′-GATCCGCTGAAGACCTGAAGACAATTTCAAGAGAATTGTCTTCAGGT
CTTCAGTTTTTTGAAA-3′

(SEQ ID NO: 6)

5′-AGCTTTTCCAAAAAACTGAAGACCTGAAGACAATTCTCTTGAAATTG
TTTCAGGTCTTCAGCG-3′
Her2:

(SEQ ID NO: 7)

5′-GATCCGGACGAATTCTGCACAATGTTCAAGAGACATTGTGCAGAATT
CGTCCCCTTTTTTGGAAA-3′

(SEQ ID NO: 8)

5′-GCTTTTCCAAAAAAGGGGACGAATTCTGCACAATGTCTCTTGAACAT
TGTGCAGAATTCGTCCG-3′
pHC_Her2 D2:

(SEQ ID NO: 9)

5′-GATCCGTATGTGAACCAGCCAGATGTTCAAGAGACATCTGGCTGGTT
CACATATTTTTTGGAAA-3′

(SEQ ID NO: 10)

5′-AGCTTTTCCAAAAAATATGTGAACCAGCCAGATGTCTCTTGAACATC
TGGCTGGTTCACATACG-3′
pHC_Her id1:

(SEQ ID NO: 11)

5′-GATCCGACGAATTCTGCACAATGGTTCAAGAGACCATTGTGCAGAAT
TCGTCCCTTTTTTGGAAA-3′

(SEQ ID NO: 12)

5′-AGCTTTTCCAAAAAAGGGACGAATTCTGCACAATGGTCTCTTGAACC
ATTGTGCAGAATTCGTCG-3′
pHC_Her id2:

(SEQ ID NO: 13)

5′-GATCCGGACGAATTCTGCACAATGTTCAAGAGACATTGTGCAGAATT
CGTCCCCTTTTTTGGAAA-3′

(SEQ ID NO: 14)

5′-AGCTTTTCCAAAAAAGGGGACGAATTCTGCACAATGTCTCTTGAACA
TTGTGCAGAATTCGTCCG-3′
pHC_Her id4:

(SEQ ID NO: 15)

5′-GATCCAGACGAAGCATACGTGATGTTCAAGAGACATCACGTATGCTT
CGTCTAATTTTTTGGAAA-3′

(SEQ ID NO: 16)

5′-AGCTTTTCCAAAAAATTAGACGAAGCATACGTGATGTCTCTTGAACA
TCACGTATGCTTCGTCTG-3′

All hairpins contain the 9 bp sequence TTCAAGAGA (SEQ ID NO: 17) as loop structure. The pHC vectors can be employed for assays requiring transient transfections as well as generation of stable transfectants silencing the desired target gene.

### EXAMPLE 2

Knock-Down Efficiency of HER2 Protein that is Expressed on the Cell Surface

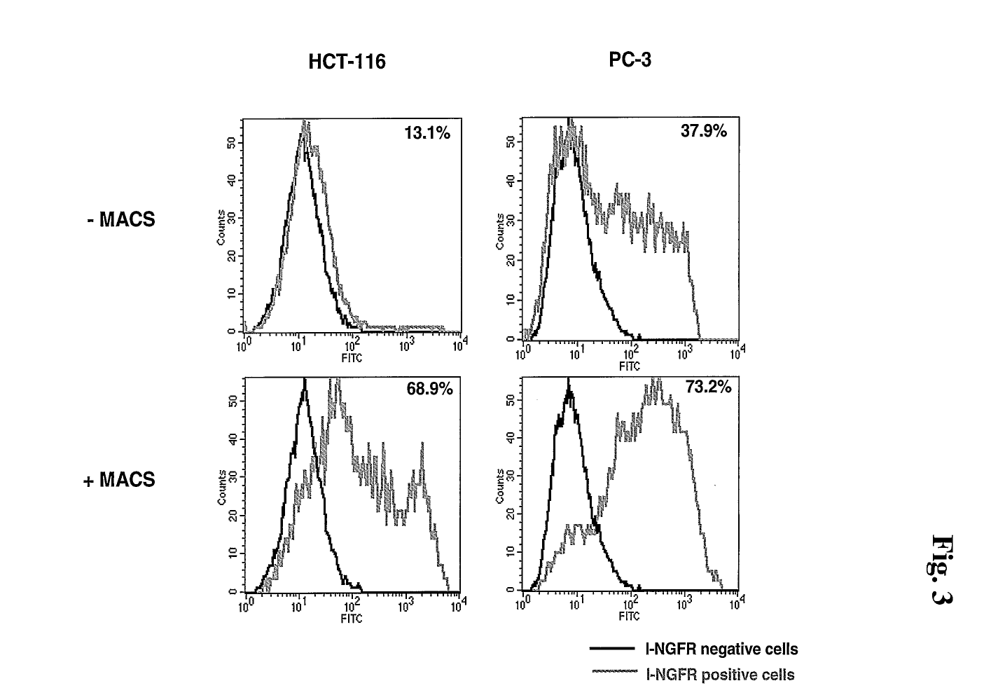

SKBR3 cells were transfected with 4 different pHC_Her2 constructs encoding shRNAs directed against HER2. Transfection with pHC luc targeting luciferase, which is not expressed in these cells, served as negative control. HER2 expression in pHC_luc and pHC_Her Fugene 6 (Roche Diagnostics) transfected cells was determined by staining the cells for HER2 as well as for 1-NGFR followed by flow cytometric analysis. Gating on 1-NGFR positive cells enables exclusive analysis of the transfected cell population. Results are shown in FIG. 2. In the histogram plots black histograms represent HER2 expression of non-transfected (1-NGFR negative) cells and grey histograms represent HER2 expression transfected (1-NGFR positive) cells recorded from the same sample. The dashed graphs in the histogram plot represent isotype control staining. Numbers on the x-axis and above the histograms indicate mean fluorescence intensities of HER2 expression; FL2-Height: HER2 expression (indirect immunofluorescence staining 1^{st} AB: rhu MAB 2C4 (Genentech), 2^{nd} AB: anti-human IgG-PE conjugate (Caltag)); 1-NGFR expression was detected with an FITC-labeled anti-1-NGFR antibody (Boehringer Mannheim). pHC_Her D2, pHC_Her id1, pHC_Her id2 and pHC_Her id4 represent 4 different vector constructs encoding 4 different shRNAs against HER2.

Successful silencing of HER2 correlated very well with 1-NGFR expression as determined by flow cytometry analysis. Thus, proteins expressed on the cell surface are amenable to the inventive high content RNAi approach exploiting pHC_vectors, which encode 1-NGFR and shRNA expression cassettes.

### EXAMPLE 3

### Enrichment of 1-NGFR-Positive Cells by Magnetic Cell Separation (MACS) in Transient Transfection Experiments

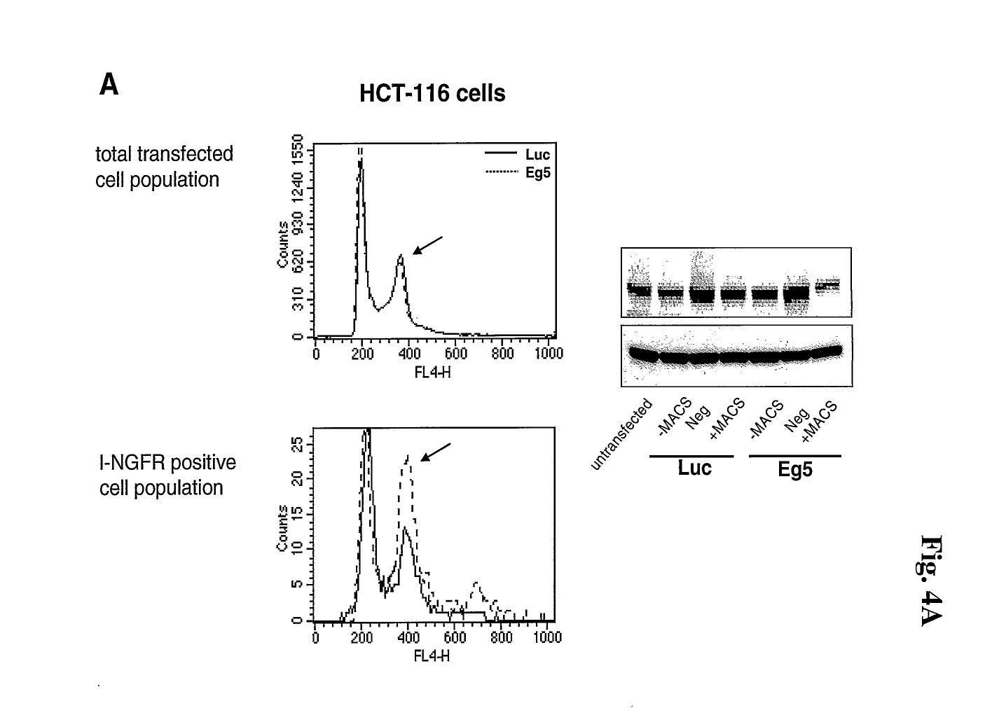

The MACSelect 1-NGFR System (Miltenyi Biotech) was used to enrich 1-NGFR positive cells 96 hrs after transfection according to example 2 of pHC_luc. In the histograms (FIG. 3), 1-NGFR expression of non-transfected cell populations (black curves) was compared to 1-NGFR expression before MACS sorting (upper panel, grey curves) and after MACS sorting (lower panel, grey curves) of pHC_luc transfected cell populations, respectively. Here, 1-NGFR expression was determined by flow cytometric detection of <Anti-1-NGFR>-Microbead labeled cells applying <Anti-Mouse>-Alexa488 Ab (Molecular Probes). The number in the histograms indicates the percentage of Microbead labeled cells before and after magnetic cell separation.

Thus, application of 1-NGFR as reporter offers the unique option to enrich silenced cells via magnetic cell sorting.

### EXAMPLE 4

### Knock-Down Efficiency in Magnetic Sorted Compared to Non-Sorted Cells and Phenotypic Alterations Induced by Silencing of Cytoplasmic Protein eg5

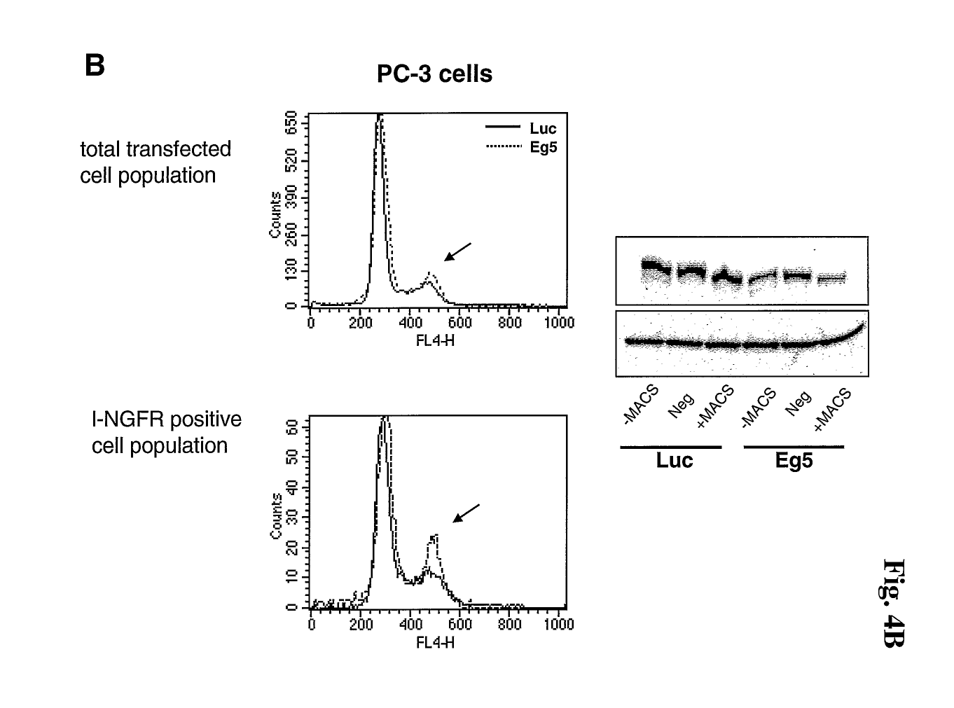

A: The kinesin protein eg5 plays an important role in mitotic spindle assembly. Consistently knocking-down eg5 expression in actively proliferating cells leads to induction of G2/M arrest followed by apoptosis. Cell cycle analysis of HCT-116 cells was performed 96 hrs after Fugene 6 transfection (Roche Diagnostics) of pHC_luc (control) and pHC_eg5 by double-staining of cells with FITC labeled anti-1-NGFR antibody and the DNA dye Hoechst 33342. The histogram plots of FIG. 4a display cell cycle distribution of cells transfected with pHC_luc (black curves) or pHC_eg5 (dashed curves) without and with gating on 1-NGFR positive cells (upper and lower panel, respectively). The arrows in the histograms indicate G2/M phase elevation of cells transfected with pHC eg5. Western Blot analysis of eg5 protein expression in HCT-116 cells before and after MACS sorting (see example 3). Total protein lysates from cells were obtained 96 hrs after transfection with pHC_luc (negative control) and pHC_eg5. The samples were named as followed: —MACS: eg5 expression before enrichment of pHC transfected cells in the original sample after transfection; Neg: eg5 expression of flow-through cell population (non-transfected, 1-NGFR negative cells do not bind to MACS column); +MACS: eg5 expression of enriched cell population (transfected, 1-NGFR positive cell fraction eluted from MACS column).

B: The identical analysis as described in example 4 A was performed in PC3 cells, which are very good transfectable and is shown in FIG. 4b. But even in this cell line gating on 1-NGFR-positive cells improves the outcome of phenotype analysis profoundly. In parallel, enrichment of 1-NGFR positive cells by MACS improves the detection of knock-down efficiency by Western Blot.

Thus, magnetic sorting based on 1-NGFR expression enables detection of knock-down efficiency already in transiently transfected cells by various biochemical methods (such as RT-PCR, Northern Blotting, Western Blotting).

### EXAMPLE 5

### Phenotypic Alterations Induced by Silencing of Cytoplasmic Protein eg5 in Transient Transfection Assays

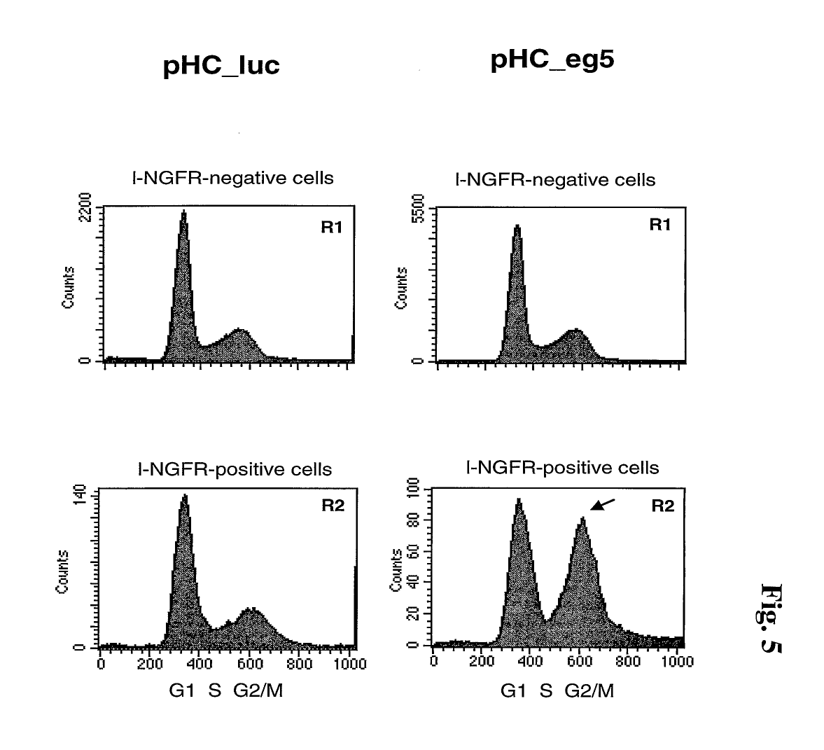

Cell cycle analysis of HCT-116 cells was performed 72 hrs after transfection of pHC_luc (control) and pHC_eg5 as in example 4 by double-staining of cells with FITC labeled anti-1-NGFR antibody and the DNA dye Hoechst 33342. In FIG. 5, 1-NGFR expression is plotted as log10 versus linear scale of Hoechst 33342 fluorescence intensity. R1: non-transfected (1-NGFR-negative) cells; R2: transfected (1-NGFR positive) cells. The arrow indicates G2/M phase elevation of cells transfected with pHC_eg5.

It can be concluded that proteins located in the cytoplasm are amenable according to the high content RNAi exploiting pHC_vectors of the present invention, which encode 1-NGFR and shRNA expression cassettes.

### EXAMPLE 6

### Multiparametric Analysis of Phenotypic Alterations Induced by Silencing of eg5 in Transient Transfection Assays

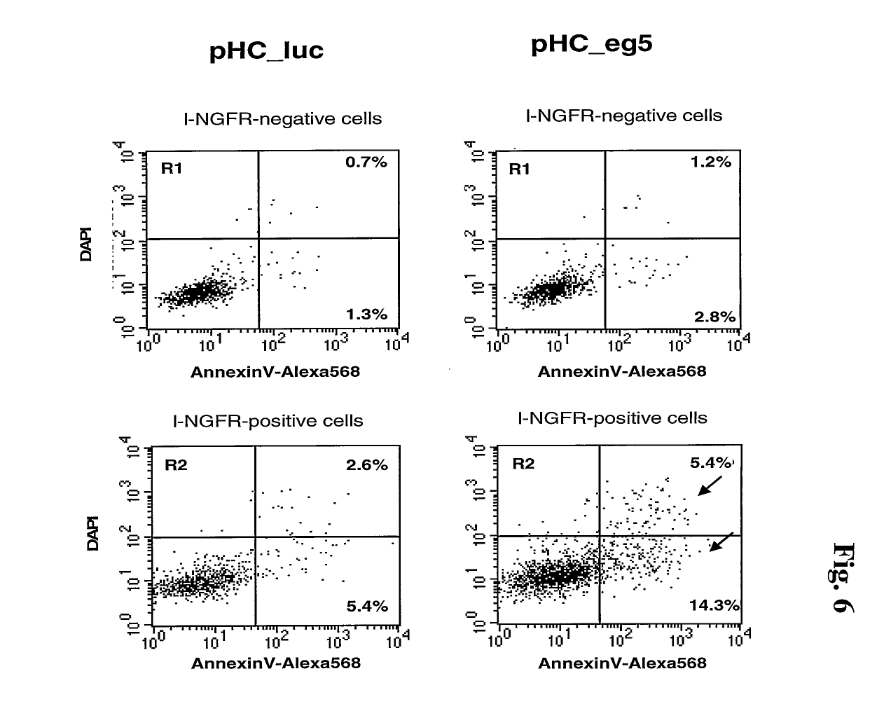

Detection of apoptotic cells transfected as in example 4 by double staining for AnnexinV (Roche) and DAPI (Roche) was combined with 1-NGFR detection to determine the percentage of apoptosis in eg5 silenced cells 72 hrs post-transfection. AnnexinV positive cells were scored as apoptotic, AnnexinV/DAPI double stained cells as apoptotic-necrotic and DAPI positive cells as necrotic. Results are shown in FIG. 6. Arrows indicate induction of apoptosis, followed by necrosis in 1-NGFR positive (=transfected) cells silenced for eg5 expression. R1: non-transfected (1-NGFR negative) cells; R2: transfected (1-NGFR positive) cells.

In dying cells, where membrane integrity is impaired, cytoplasmic proteins such as EGFP are easily lost in contrast to 1-NGFR, which is bound to the cell membrane. This indicates that both apoptotic and apoptotic-necrotic cells stain positive for 1-NGFR expression. Thus, the use of 1-NGFR containing pHC_vectors facilitates analysis of targets which induce apoptosis upon silencing.

### LIST OF REFERENCES

Barton, G. M., and Medzhitov, R., Proc. Natl. Acad. Science USA 99 (2002) 14943-14945
Brummelkamp, T. R., and Bernards, R., Nat. Rev. Cancer 3 (2003) 781-789
Caplen, N. J., Gene Ther. 11 (2004) 1241-1248
Chalfie, M., and Kain, S., (eds), in Green Fluorescent Protein: properties, applications and protocols, Wiley-Liss, New York, 1998
Dykxhoorn, D., et al., Nat. Rev. Mol. Cell Biol. 4 (2003) 457-467
EP 1 114 784
EP 1 144 623
Harvey, D. M., and Caskey, C. T., Curr. Opin. Chem. Biol. 2 (1998) 512-518
Harvey, K. J., et al., Cytometry 43 (2001) 273-278
Kojima, S., et al., Biotechniques 36 (2004) 74-79
Machl, A. W., et al., Cytometry 29 (1997) 371-374
Phillips, K., et al., Nat. Med. 2 (1996) 1154-1156
Singer, O., et al., Proc. Natl. Acad. Sci. USA 15 (2004) 5313-5314
Tuschl, T., Nat. Biotechnol. 20 (2002) 446-448
U.S. Pat. No. 6,506,559
Ventura, A., et al., Proc. Natl. Acad. Sci. USA 101 (2004) 10380-10385
WO 03/93430

1. A method for inactivation of expression of a gene in a eucaryotic cell comprising
a) transforming a eucaryotic cell with DNA comprising an expression cassette for expression of a cell surface protein and an expression cassette for expression of a RNAi compound, said compound being capable of inactivating expression of said gene, wherein said expression cassette for expression of a cell surface protein and said expression cassette for expression of a RNAi compound are located on the same vector DNA,
b) enrichment and/or selection of cells which express said cell surface protein.

2. A method according to claim 1, wherein said RNAi compound is a RNA with a hairpin conformation.

3. The method of claim 1, wherein said enrichment and/or selection of cells which express said cell surface protein is performed by means of cell sorting.

4. A vector comprising an expression cassette for expression of 1-NGFR and an expression cassette for expression of a RNAi compound, said compound being capable of inactivating expression of a gene in a eukaryotic cell.

5. A composition comprising
a) a transfection reagent, whereby said transfection is a lipofection reagent such as FuGENE 6. X-tremeGENE or Lipofectamine™; and
b) DNA comprising an expression cassette for expression of a cell surface protein and an expression cassette for expression of a RNAi compound, wherein said expression cassette for expression of a cell surface protein and said expression cassette for expression of a RNAI compound are located on the same vector DNA.

6. A kit comprising
a) a vector comprising an expression cassette for expression of a cell surface protein and an expression cassette for expression of a RNAi compound;
b) a monoclonal antibody or a polyclonal antiserum directed against said cell surface protein, and
c) a transfection reagent, whereby said transfection reagent is a lipofection reagent such as FuGENE 6, X-tremeGENE or Lipofectamine™.

7. A kit according to claim 6, wherein said antibody is labeled with a detectable entity or covalently bound to a solid support.

8. A method for the selection of cells transfected with RNAi constructs for inactivation of expression of a gene comprising
a) transforming a eukarvotic cell with DNA comprising an expression cassette for expression of a ell surface protein and an expression cassette for expression of a RNAi compound, said compound being capable of inactivating expression of said gene, wherein said expression cassette for expression of a cell surface protein and said expression cassette for expression of a RNAi compound are located on the same vector DNA, and
b) enrichment and/or selection of cells which express said cell surface protein.


SEQUENCE LISTING (17 sequences)

SEQ ID NO: 1 - DNA, 34 bp, Artificial Sequence
  Description of Artificial Sequence Synthetic primer
cgcctcgagtccctgtggaatgtgtgtcagttag

SEQ ID NO: 2 - DNA, 32 bp, Artificial Sequence
  Description of Artificial Sequence Synthetic primer
cgcaagcttgctggccttttgctcacatgttc

SEQ ID NO: 3 - DNA, 60 bp, Artificial Sequence
  Description of Artificial Sequence Synthetic oligonucleotide
gatccgcttacgctgacttcgattcaagagatcgaagtactcagcgtaagttttttggaa

SEQ ID NO: 4 - DNA, 64 bp, Artificial Sequence
  Description of Artificial Sequence Synthetic oligonucleotide
agcttttccaaaaaacttacgctgagtacttcgatctcttgaatcgaagtactcagcgtaagcg

SEQ ID NO: 5 - DNA, 63 bp, Artificial Sequence
  Description of Artificial Sequence Synthetic oligonucleotide
gatccgctgaagacctgaagacaatttcaagagaattgtcttcaggtcttcagttttttgaaa

SEQ ID NO: 6 - DNA, 63 bp, Artificial Sequence
  Description of Artificial Sequence Synthetic oligonucleotide
agcttttccaaaaaactgaagacctgaagacaattctcttgaaattgtttcaggtcttcagcg

SEQ ID NO: 7 - DNA, 65 bp, Artificial Sequence
  Description of Artificial Sequence Synthetic oligonucleotide
gatccggacgaattctgcacaatgttcaagagacattgtgcagaattcgtccccttttttggaaa

SEQ ID NO: 8 - DNA, 64 bp, Artificial Sequence
  Description of Artificial Sequence Synthetic oligonucleotide
gcttttccaaaaaaggggacgaattctgcacaatgtctcttgaacattgtgcagaattcgtccg

SEQ ID NO: 9 - DNA, 64 bp, Artificial Sequence
  Description of Artificial Sequence Synthetic oligonucleotide
gatccgtatgtgaaccagccagatgttcaagagacatctggctggttcacatattttttggaaa

SEQ ID NO: 10 - DNA, 64 bp, Artificial Sequence
  Description of Artificial Sequence Synthetic oligonucleotide
agcttttccaaaaaatatgtgaaccagccagatgtctcttgaacatctggctggttcacatacg

SEQ ID NO: 11 - DNA, 65 bp, Artificial Sequence
  Description of Artificial Sequence Synthetic oligonucleotide
gatccgacgaattctgcacaatggttcaagagaccattgtgcagaattcgtcccttttttggaaa

SEQ ID NO: 12 - DNA, 65 bp, Artificial Sequence
  Description of Artificial Sequence Synthetic oligonucleotide
agcttttccaaaaaagggacgaattctgcacaatggtctcttgaaccattgtgcagaattcgtcg

SEQ ID NO: 13 - DNA, 65 bp, Artificial Sequence; identical to SEQ ID NO: 7
  Description of Artificial Sequence Synthetic oligonucleotide

SEQ ID NO: 14 - DNA, 65 bp, Artificial Sequence
  Description of Artificial Sequence Synthetic oligonucleotide
agcttttccaaaaaaggggacgaattctgcacaatgtctcttgaacattgtgcagaattcgtccg

SEQ ID NO: 15 - DNA, 65 bp, Artificial Sequence
  Description of Artificial Sequence Synthetic oligonucleotide
gatccagacgaagcatacgtgatgttcaagagacatcacgtatgcttcgtctaattttttggaaa

SEQ ID NO: 16 - DNA, 65 bp, Artificial Sequence
  Description of Artificial Sequence Synthetic oligonucleotide
agcttttccaaaaaattagacgaagcatacgtgatgtctcttgaacatcacgtatgcttcgtctg

SEQ ID NO: 17 - DNA, 9 bp, Artificial Sequence
  Description of Artificial Sequence Synthetic oligonucleotide
ttcaagaga